(19) United States
(12) Patent Application Publication
(10) Pub. No.: US 2007/0207167 A1
(43) Pub. Date: Sep. 6, 2007

(54) Immunologically enhanced recombinant vaccines

(76) Inventors: Carl R. Merril, Bethesda, MD (US); Biswajit Biswas, Germantown, MD (US); Michael McKinstry, Bethesda, MD (US)

Correspondence Address: M. Elisa Lane;Hygea BioPharma, Inc., 207 Perry Parkway, Suite 2, Gaithersburg, MD, 20877, US

(21) Appl. No.: 11/645,817

(22) Filed: Dec. 26, 2006

Related U.S. Application Data
(60) Provisional application No. 60/753,506, filed on Dec. 23, 2005.
(60) Provisional application No. 60/783,278, filed on Mar. 17, 2006.

(51) Int. Cl. A61K 39/12; C12N 15/86; C12N 7/00

(52) U.S. Cl. 424/199.1; 435/235.1; 435/456

(57) ABSTRACT

The present invention is directed to recombinant vaccines, based on a phage vector system, which enhance immunological response and allow rapid construction and deployment of vaccines.

This application claims priority to provisional applications Serial Nos. 60/753,506, filed Dec. 23, 2005, and 60/783,278, filed Mar. 17, 2006, the contents of each being incorporated by reference.

### FIELD OF THE INVENTION

The present invention is related to the field of recombinant vaccines, which are genetically engineered to enhance the host's immune response thereto. The invention is directed to phage vector vaccines, which are engineered to display one or more exogenous peptides on its outer coat such as to target and maximize the interaction of the phage with professional immune cells such as macrophages and dendritic cells. More specifically, the invention provides multi-component genetic vaccines, which contain one or more genes coding for immunogens of interest under the control of mammalian expression promoter(s) as well as one or more genes for immune cell-targeting peptides that will be expressed on the phage capsid. Upon delivery to the host, the vaccines engineered as such will target and infect the professional immunes cells, where upon transfection, such cells will express and process the immunogen peptides of interest. In this manner, the processed immunogen can be efficiently presented to the antibody-producing cells, resulting in an enhanced protective response.

### BACKGROUND OF THE INVENTION

For years, the use of bacteriophage for vaccine purposes has been proposed. Phages were first used to introduce specific genes into mammalian cells in the early 1970s. However, typically in such cases the phage is engineered to express a protein on its surface (often as a fusion product of a major phage coat protein and the antigen of interest) in order to induce antibody production. Thus, the fusion protein expressed is the intended immunogen of the vaccine. For example, see De Berardinis et al., (2000) Phage display of peptide epitopes from HIV-1 elicits strong cytolytic responses. Nat Biotechnol. 18: 873-876; Ying Wan et al., (2001) Induction of hepatitis B virus-specific cytotoxic T lymphocytes response in vivo by filamentous phage display vaccine, Vaccine 19: 2918-1923; Yuzhang Wu et al., (2002) Phage display particles expressing tumor-specific antigens induce preventive and therapeutic anti-tumor immunity in murine p815 model, International Journal of Cancer, 98: 748-753; Ying Wan et al., (2005) Cross-presentation of phage particle antigen in MHC class II and endoplasmic reticulum marker-positive compartments, European Journal of Immunology, 35: 2041-2050.

In addition, there are a few reports in the literature that disclose the use of a eukaryotic promoter-driven vaccine gene along with a displayed fusion protein. While this technology approaches the concept of the present invention, none of the references suggest the independent use of a phage-displayed protein designed to maximize an antibody response by enhancing uptake by professional immune cells as in the present invention. See further, Merril et al., (1971) Bacterial virus gene expression in human cells, Nature 233: 398400; and Horst et al., (1975) Gene transfer to human cells: transducing phage Lambda plac gene expression in GM1-gangliosidosis fibroblasts, Proc. Natl. Acad. Sci. USA 72: 3531-3535.

March et al., (2004) Genetic immunisation against hepatitis B using whole bacteriophage particles. Vaccine, 22: 1666-1671, which teaches: “Mice and rabbits have been vaccinated with whole bacteriophage lambda particles containing a DNA vaccine expression cassette under the control of the CMV promoter (enhanced green fluorescent protein [lambda-EGFP] or hepatitis B surface antigen [lambda-HBsAg]). Mice were vaccinated twice intramuscularly (i.m.) with 5×10(9) of lambda-EGFP phage (containing 250 ng DNA) and exhibited specific anti-EGFP responses 28 days post-vaccination. Rabbits were vaccinated i.m. with 4×10(10) of lambda-HBsAg phage (2 microg DNA) or recombinant HBsAg protein. Following two vaccinations with lambda-HBsAg, one out of four rabbits exhibited high level anti-HBsAg responses (comparable to those seen using the recombinant HBsAg protein). Following a third vaccination with lambda-HBsAg, all four rabbits showed similar high level responses which have not decreased after more than 6 months. High anti-phage responses were observed in all animals following the first immunization with lambda-HBsAg, indicating that a high antibody titre against the phage carrier did not prevent a subsequent immune response against the DNA vaccine component. Compared to results in mice using equivalent lambda-HBsAg doses, anti-HBsAg responses were much higher in rabbits, which could indicate a swamping effect in mice. Since phage lambda DNA is approximately 50 kb in size (tenfold larger than most plasmid vectors used for naked DNA immunisation), a comparable dose of phage lambda DNA given as intact phage particles actually delivers tenfold less vaccine DNA on a per gene copy (molar) basis. Thus the efficiency of the technique may be even higher than the data at first suggests.”

Also, Clark et al. (2004) Bacteriophage-mediated nucleic acid immunisation. EMS Immunol Med Microbiol. 40: 21-26, which discloses: Whole bacteriophage lambda particles, containing reporter genes under the control of the cytomegalovirus promoter (P(CMV)), have been used as delivery vehicles for nucleic acid immunisation. Following intramuscular injection of mice with lambda-gt11 containing the gene for hepatitis B surface antigen (HBsAg), anti-HBsAg responses in excess of 150 mlU per ml were detected. When isolated peritoneal macrophages were incubated with whole lambda particles containing the gene for green fluorescent protein (GFP) under the control of P(CMV), GFP antigen was detected on the macrophage surface 8 hours later. Results suggested that direct targeting of antigen-presenting cells by bacteriophage ‘vaccines’ may occur, leading to enhanced immune responses compared to naked DNA delivery.

In Clark et al., (2004) Bacterial viruses as human vaccines? Expert Rev Vaccines 3: 463476, the authors note “that phage are viruses of bacteria, consisting of nucleic acid packaged within a protein coat. In eukaryotic hosts, phages are unable to replicate and in the absence of a suitable prokaryotic host, behave as inert particulate antigens. In recent years, work has shown that whole phage particles can be used to deliver vaccines in the form of immunogenic peptides attached to modified phage coat proteins or as delivery vehicles for DNA vaccines, by incorporating a eukaryotic promoter-driven vaccine gene within their genome. While both approaches are promising by themselves, in future there is also the exciting possibility of creating a hybrid phage combining both components to create phage that are cheap, easy and rapid to produce and that deliver both protein and DNA vaccines via the oral route in the same construct.”

Further, Jepson et al., (2004) Bacteriophage lambda is a highly stable DNA vaccine delivery vehicle. Vaccine, 22: 2413-2419, which reports: The stability of whole bacteriophage lambda particles, used as a DNA vaccine delivery system has been examined. Phage were found to be highly stable under normal storage conditions. In liquid suspension, no decrease in titre was observed over a 6-month period at 4 and −70° C., and phage stability was unaffected by freeze/thawing. When stored at −70° C., desiccated phage appeared to be stable in the absence of stabilizers. When phage lambda was diluted into water, a marginal loss in titre was observed over a 2-week period. Over a 24 h period, liquid phage suspensions were stable within the pH range pH 3-11, therefore oral administration of bacteriophage DNA vaccines via drinking water may be possible.

There remains a need in the art for a vaccine strategy that will effectively immunize against any one of numerous pathogenic entities of interest, and is relatively easy and inexpensive to prepare.

### BRIEF DESCRIPTION OF THE DRAWINGS

FIG. 1. Depicts plasmid (“pCMV-I”) containing the CMV promoter, the cloned antigen gene, and an SV40 polyA signal. See Example 1.

FIG. 2. Depicts the cloning of the antigen gene/CMV promoter construct of FIG. 1 into the EcoR1 site of plasmid pVCDcDL3, and the schematic diagram of the resulting recombinant plasmid. See Example 1.

FIG. 3. Depicts the cloning of two copies of the antigen gene: one associated with a mammalian promoter and the other as a fusion gene with a gene for a major coat protein of the phage (the D capsid gene), and the resulting construct. See Example 1.

FIG. 4. Depicts lambda phage DL1 containing the construct loxPwt-lacZalpha-loxP511 inserted into its genome between genes J and Int. Recombination will occur between the lox sites of the plasmid and lox sites of phage DL1, resulting in the introduction of plasmid DNA into the phage genome.

FIG. 5. Cloning of VP2 antigen in PCMV plasmid. The VP2 antigen gene is PCR amplified by using two modified primers F1 and R1. The sequence information of the F1 primer is obtained from the upstream and downstream region of the VP2 gene sequence of IBDV. Recombinant clone plBDVP2 containing VP2 gene is used as template for PCR amplification. Amplified product is cloned in pCMV-Script plasmid and designated as pCMV-1. Subsequent amplification of VP2 antigen along with CMV promoter is accomplished by using two modified primers designated as F2 and R2. The amplified product (VP2 gene along with upstream CMV promoter) is designated as construct 1. The maps are not to scale.

FIG. 6. VP2 gene with upstream CMV promoter (construct I) is restriction digested and cloned in EcoRI site of pVCDcDL3 plasmid. The resulting recombinant is designated as pVCD-1. The maps are not to scale.

FIG. 7. Cloning of VP2 gene in recombinant pVCD-1 plasmid. VP2 gene is amplified from plBDVP2 recombinant plasmid by using two modified primers designated as F3 and R3. Amplified product is restriction digested and cloned in SmaI site of recombinant plasmid pVCD-1 and designated as recombinant plasmid pVCD-2. The maps are not to scale.

FIG. 8. Homologous recombination of donor plasmid pVCD-2 with recipient phage vector Lambda (A) DL1 phage. Only some of the lambda genes are shown. The unique SmaI site in the lambda genome used for cloning is shown. lacZa,DNA cassette comprised of lacPO, RBS and the first 58 codons of lacZ. Generated recombinant phage is designated as Lambda-VP2 which contains two separate insert of VP2 antigen genes. One insert is fused with GpD head protein gene of lambda to produce GpD-VP2 fusion on lambda capsid. Other insert simply inserted into non essential region of lambda genome under control of CMV promoter. The maps are not to scale.

FIG. 9. The construction of a lambda phage containing a dendritic cell-targeting peptide is performed using the methods described in Example 3. See also Example 8.

FIG. 10. The plasmid is designated pVCD-3/pDual GC. See Example 8.

FIG. 11. The recombinant plasmid, designated as pVCD-3/pDual GC/Org plasmid. See Example 8.

### DETAILED DESCRIPTION OF THE INVENTION

An object of the present invention is to overcome the disadvantages found in the recombinant and proposed phage-based vaccines of the art.

Thus, the present invention is directed to a recombinant bacteriophage, as well as an immunogenic composition comprised of a plurality of such bacteriophage, which has been genetically engineered to express at least two components: (1) a gene, or genes, encoding immunogenic epitope(s) of one or more antigens of interest, which is/are capable of inducing antibodies in mammals and which is/are operably connected to a mammalian expression promoter; (2) and a gene, or genes, operably connected to a bacterial promoter allowing expression of a fusion peptide of a phage coat protein and an immune-stimulating peptide, such as to express a fusion peptide on the phage coat that will allow for the “professional first response” immune cells, such as dendritic cells, in the mammalian host to be preferentially targeted when an immunogenic composition of the modified phage is delivered to the host in need.

The engineered phage thus provides on its surface an immune-stimulation to the professional immune cells, favoring and targeting the frontline immune cell population, while concurrently allowing such cells to uptake the phage and express the immunogen(s) of interest, resulting in a protective response in the mammal to the undesirable foreign matter. The immunogenic composition of the present invention is applicable to the protection of a mammal against foreign microbes of any kind, as well as to elicit an immune response to undesirable cells in the body, such as cancer cells.

Bacteriophage DNA vaccines offer several advantages: they do not contain antibiotic resistance genes, they offer a large cloning capacity (approximately 15 kb), the DNA is protected from environmental degradation, they offer the potential for oral delivery, and large-scale production is cheap, easy and extremely rapid.

Further aspects of the present invention include the processes of preparing the recombinant phage as well as methods of using the resulting phage for vaccination against pathogens (which for the purposes of this disclosure include cancer antigens).

The invention is based on the following observations and discoveries.

Antigens first react with what are frequently referred to as “professional” immune cells, which pass the antigens to activated T cells, which in turn present them to B cells for antibody production, in accordance with the diagram below.

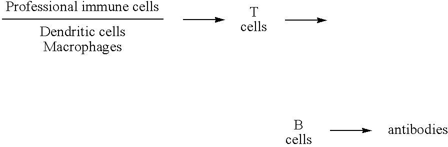

When vaccines are injected into the skin, the dendritic cells at the site of injection are immature and not efficient at presenting antigens to naive T cells. However, there are a special class of such cells, the Langerhan's cells, that are actively phagocytic and migrate to regional lymph nodes where they normally express B7 glycoproteins, which co-stimulate additional naive T cells.

Dendritic cells may be infected by viruses, such as the subject bacteriophages, which will either bind to any of several molecules on the cell surface and then are taken in or are engulfed, but not destroyed, by the cells. The viruses will synthesize their proteins in the dendritic cells (in the present invention, genes with mammalian promoters as well), which leads to cell surface expression of the viral peptides for presentation to the T cells. It is also noted that dendritic cells can also take up external protein directly for presentation to T-cells. Mononuclear phagocytes or macrophages can behave in a similar manner to the dendritic cells in the presentation of antigens to T cells. It is critical to co-stimulate the dendritic cells, however, because antigen recognition in the absence of co-stimulation can inactivate naive T cells inducing a state known as anergy (just the opposite of what is needed to achieve with vaccination).

Thus, at its essence, in addition to one or more immunogenic epitope genes, which are operably attached to mammalian promoters and expressed in the professional immune cells, the present invention requires “immunogenic enhancer” genes to be expressed by the engineered phage on the phage surface or that are operably attached to mammalian promoters and expressed on the surface of the professional immune cells. Such a system ensures an adequate immune-protective response by the host, something that has been elusive with past attempts at phage vaccines.

(For purposes of the present disclosure the terms “peptide,” “polypeptide,” and “protein” are largely interchangeable.)

There are numerous possibilities for genes expressing immunogenic enhancers, which will serve to stimulate the frontline immunogenic response in the vaccinated subject, and the present invention is not limited to only certain proteins.

The present invention provides for a polynucleotide that expresses a peptide that has a modulatory effect on the immune response desired by a recombinant phage vaccine, either directly (i.e., as an immunomodulatory peptide/phage coat fusion molecule) or indirectly (i.e., upon translation of the polynucleotide to create an immunomodulatory peptide in a professional immune cell).

As examples of such peptides are CpG-rich polynucleotide sequences, polynucleotide sequences that encode a costimulator (e.g., B7-1, B7-2, CD1, CD40, CD154 (ligand for CD40), CD150 (SLAM)), or a cytokine, some of which are described more fully below.

The B7 glycoprotein gene: by placing the gene for a B7 molecule (a glycoprotein that stimulates the clonal expansion of naive T cells) in a phage of the present invention operably linked to a mammalian promoter, the molecule is expressed by the phage DNA in the professional immune cells on the surface thereof, which results in a better or stronger T cell response and, in turn, a better B cell antibody production to the concurrently expressed antigen. B7 is also known as B7.1 (CD80) or B7.2 (CD86).

Vaccine antigen gene coupled to a CTLA-4 (CD152) gene: The CTLA gene encodes the receptor for B7.1. It has previously been used with DNA vaccines, and has been shown to selectively bind the expressed proteins to the antigen-presenting cells carrying B7.

Vaccine antigen gene coupled to signal peptide that targets a lysosomal-associated membrane protein to lysosomes and endosomes: This genetically engineered system will direct the vaccine antigen directly to the intracellular compartments where the antigens are cleaved to peptides before binding to MHC class II molecules for display to T cells.

Heat shock protein genes: The activation of these genes inside the dendritic cells that take up the phage provides for intracellular chaperones for the vaccine antigenic peptide, which will facilitate the antigen's movement to the surface membranes of the dendritic cells for antigen presentation to T cells.

Granulocyte-macrophage colony-stimulating factor (GM-CSF) gene: This protein enhances the production of macrophages and dendritic cells. The use of this gene in the engineered phage may be as a fusion product of a major phage capsid gene for expression on the phage surface for immunogenic enhancement purposes, but also as operably attached to a mammalian promoter and produced by the dendritic cell for cell surface presentation thereof.

Further, immunogenic enhancers in the present invention include peptides associated with bacterial endotoxins and exotoxins, and cytokines and interleukins such as IL-15 and IL-2.

In the recombinant system of the present invention whereby peptides are to be expressed as fusion products of a major phage capsid protein to achieve immunogenic enhancement are mentioned the following for purposes of illustration:

IL-2 (interleukin-2): This protein is a cytokine, normally produced by activated T-cells. When the protein encounters an IL-2 receptor on a T cell, it causes the T cell to divide and differentiate into armed effector T-cells. In view of the fact that there are two types of T-cells, CD4 T cells and CD8 T cells, this system could be used for microbe vaccination purposes or, since CD8 T cells are cytotoxic or killer T cells it's useful also for anti-tumor therapy.

In the case of the CD4 cells the situation is somewhat more complex, as they can differentiate into TH1 or TH2 cells as illustrated below, and such is taken into consideration when engineering a phage for the desired response.

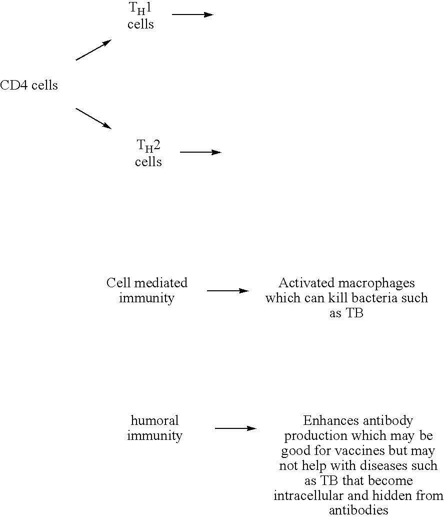

Granulocyte-macrophage colony-stimulating factor (GM-CSF) gene: This gene when expressed in the host cell will increase the production of macrophages and dendritic cells.

Genes for fimbrial proteins of Salmonella typhimurium: These proteins play a key role in the binding of bacteria to mucosal M cells, which are involved in the immune response in the gastrointestinal tract. Such a system is ideal for an orally delivered vaccine.

In a recent PNAS paper, central memory CD8 T cells (TCM) and effector memory CD8 T cells (TEM) are found in humans and mice to be activated by IL-15. These cells are particularly important, as adoptively transferred TCM exhibited a potent in vivo recall response when combined with tumor-antigen vaccination and exogenous IL-2, leading to the eradication of large established tumors. TCM have been also been shown to be superior to TEM in conferring protective immunity against viral or bacterial challenge. See Klebanoff et al. PNAS, Jul. 5, 2005, vol. 102, no. 27, pp. 9571-9576.

The protein, Vcam-1, the ligand of VIa-4, is not expressed on normal blood vessels, but it is upregulated in tumor neovessels 27-30. This protein has been show to attract T cells in a recent study demonstrating its importance in experimental treatment of melanoma. See Meunier et al., “T cells targeted against a single antigen can cure solid tumors,” Nature Medicine, 11(11), pp.1222-1229 (2005).

The above-noted list of immune enhancer elements is far from exhaustive, and the literature is replete with information on numerous genetic sequences and fusion genes containing them, even specifically to enhance an immune response, which the person of ordinary skill in the art would have readily at hand to accomplish the practice of the present invention. Thus, the present invention is not directed to these genes and their use in immunogenic enhancement per se, but their use in the subject genetically engineered phage, which has not been contemplated until now. That is, these elements have been used in DNA vaccine formats, for instance for cancer immunotherapy. Accordingly, the present invention relies on these previous disclosures for their teachings of identifying, obtaining and cloning or fusing these genetic elements into phage for the purposes of the entire recombinant vaccines contemplated herein. Moreover, the choice of immunogenic enhancer elements for the present invention depends in part on the antigen that is the subject of the vaccination, and such choice is within the knowledge of the ordinary person in the art.

Further, the present invention contemplates the use of any one of thousands of genes for peptides or proteins (or more particularly the epitopes) that will elicit an immune response to an antigen (for instance, of a endogenous tumor) or microbe of concern (which may act in a protective manner as a vaccine, or directly against the endogenous material).

Moreover, the peptides or proteins useful as antigenic elements in the present invention do not need to be as immunogenic as those that in the past have been required to elicit a sufficient immune response, due to the use of the immune enhancer elements herein.

In fact, because the present invention employs phage, which can be easily and rapidly produced, one may also use a “shotgun” approach, where a library of recombinant phage that will express hundreds or thousands of different epitopes of proteins of an undesired entity (microbes or cancer target peptides) is produced, to give a mixture of phages in a batch containing immunogenic enhancer elements in combination with a plurality of antigens. A vaccine containing such a variety of phages is leaps and bounds ahead of the normal course those in the art take to develop an adequate vaccine, which typically takes years to discover the particular antigens in a foreign object (e.g., cancer or microbe) that will effectively protect the host. Such a scenario in the current state of the vaccine art, which involves vaccine production in eggs or tissue culture, would be unthinkable as being entirely too labor-intensive and costly to be worthwhile. Phage, on the other hand, can be produced easily and quickly in large volume bacterial bioreactors, then collected by centrifugation, allowing for rapid response to sudden outbreaks of viral or bacterial disease, for instance.

Vaccines for the treatment of cancer may need to carry multiple tumor antigen genes (for expression in the dendritic cells). These genes could include: EADPTGHSY (melanoma) from MAGE-1 protein, EVDPIGHLY (lung carcinoma) from MAGE-3, EVDPIGHLY (lung carcinoma) from MAGE-3, and many others. (See Bellone, et al, Immunology Today, Vol 20, No.10, p 457462, 1999.) The genes may also be derived from human aspartyl (asparaginyl) beta-hydroxylase (HMH), a polypeptide found vastly overexpressed in malignant cells (see Wands et al., U.S. Pat. No. 6,835,370 and related patents). This list is not intended to be exhaustive, and many other antigenic cancer genes are known and available for use in the phage vaccines of the present invention.

Further, a report published in Molecular Microbiology vol. 56 (2005) pp 1-15 concerning presentations at the ASM Conference on the New Phage Biology, in Key Biscayne, Fla. in August of 2004, reports pertinent findings concerning phage used as vaccine vectors: “Research on the use of whole-phage particles as a delivery vehicle for a DNA vaccine against Yersinia pestis was presented by J. R. Clark (J. March group, Moredun Research Institute, Penicuik, UK). The gene for the V antigen, which has been shown to give protection against Y. pestis infection, was cloned into plasmid and bacteriophage vectors under the control of a eukaryotic expression cassette. The V antigen DNA vaccine which was delivered using the bacteriophage vector gave IgG2a responses significantly higher than that from the plasmid-borne vaccine, following intramuscular delivery. Interestingly, while phages delivered orally (by gavage needle) were not as efficacious as phages given by intramuscular inoculation, the orally administered phage preparation still matched the performance of the intramuscular plasmid vaccine. Similarly, λ(phage) and plasmid vectors containing the gene for the hepatitis B surface antigen (HBSAg) under the control of the eukaryotic cytomegalovirus promoter were used for intradermal vaccination in cannulated sheep. In the case of phage administrations, effective antiphage titres were found in the draining lymph after the second inoculation, along with a significant IgM and IgG anti-HBSAg response. The authors suggested that the virus-like properties of the phage particles result in them being taken up by professional antigen presenting cells (such as dendritic cells) where efficient expression of the vaccine genes can occur (Clark and March, 2004).

The capacity of phage to deliver genes in mammalian hosts was graphically demonstrated by C. Gorman-Zanghi (S. Dewhurst group, University of Rochester, N.Y.) with images of light emission from mice inoculated intradermally with λ phage carrying a luciferase gene. A similar delivery system is being used with a λ(phage) construct in which there is a C-terminal fusions between the gpD external virion protein and the IgG-binding domains of staphylococcal protein A and streptococcal protein G. Purified λ phage with both fusion types are being used in conjunction with antibodies specific for common dendritic cell receptors to target human and murine dendritic cells in vitro. Successful gene transductions are evaluated by luciferase and green fluorescent protein expression.

From this report and papers published in the recent literature it is clear that phage carrying either a luciferase gene or a green fluorescent protein gene can be used to optimize the uptake and expression of a gene in professional immune cells such as either the dendritic cells or macrophages in vivo by using fusion proteins combining a major phage capsid protein with a peptide of protein that optimizes uptake be professional cells. Once this is accomplished one can then substitute a gene of interest for the reporter genes (luciferase gene or a green fluorescent protein gene used to optimize the system). A gene(s) of interest could be one of the genes encoded in the avian flu virus or a malaria encoded gene for instance for a vaccine for flu or malaria respectively. It is noted that the teachings of Gorman-Zanghi, above, do not suggest the present invention. Gorman-Zanghi's goal was to induce antibodies to streptococcal proteins and they used the luciferase reporter to show that they were affecting the proper cells. In the present invention, a fusion phage capsid protein is used to direct the phage to the professional immune cells, and the reporter is replaced with a gene encoding an immunogenic peptide of interest.

The use of reporter genes provides a powerful tool to determine the fate of phage vaccines of the present invention in animals and humans. In addition, they can be used to optimize the eukaryotic promoter.

As noted above, Clark and March used a cytomegalovirus promoter as the eukaryotic expression promoter, but the present invention is not limited to a particular eukaryotic promoter or promoters, and any one of the many known endogenous promoters (i.e., derived from the genome of mammalian cells, such as the metallothionein promoter) or exogenous promoters available in the art may be used.

As the wildtype phage itself, there are a number of possibilities, including but not limited to filamentous bacteriophages, which include M13, f1, fd, If1, Ike, Xf, Pf1, and Pf3. They are termed filamentous because they are long, thin particles comprised of an elongated capsule that envelopes the deoxyribonucleic acid (DNA) that forms the bacteriophage genome. The F pili filamentous bacteriophage (Ff phage) infect only gram-negative bacteria by specifically adsorbing to the tip of F pili, and include fd, f1 and M13. Compared to other bacteriophage, filamentous phage in general are attractive and M13 in particular is especially attractive because: (i) the 3-D structure of the virion is known; (ii) the processing of the coat protein is well understood; (iii) the genome is expandable; (iv) the genome is small; (v) the sequence of the genome is known; (vi) the virion is physically resistant to shear, heat, cold, urea, guanidinium chloride, low pH, and high salt; (vii) the phage is a sequencing vector so that sequencing is especially easy; (viii) antibiotic-resistance genes have been cloned into the genome with predictable results (Hines et al. (1980) Gene 11:207-218); (ix) it is easily cultured and stored, with no unusual or expensive media requirements for the infected cells, (x) it has a high burst size, each infected cell yielding 100 to 1000 M13 progeny after infection; and (xi) it is easily harvested and concentrated (Salivar et al. (1964) Virology 24: 359-371). The entire life cycle of the filamentous phage M13, a common cloning and sequencing vector, is well understood. The genetic structure of M13 is well known, including the complete sequence (Schaller et al. in The Single-Stranded DNA Phages eds. Denhardt et al. (NY: CSHL Press, 1978)), the identity and function of the ten genes, and the order of transcription and location of the promoters, as well as the physical structure of the virion (Smith et al. (1985) Science 228:1315-1317; Raschad et al. (1986) Microbiol Dev 50:401427; Kuhn et al. (1987) Science 238:1413-1415; Zimmerman et al. (1982) J Biol Chem 257:6529-6536; and Banner et al. (1981) Nature 289:814-816). Because the genome is small (6423 bp), cassette mutagenesis is practical on RF M13 (Current Protocols in Molecular Biology, eds. Ausubel et al. (NY: John Wiley & Sons, 1991)), as is single-stranded oligonucleotide directed mutagenesis (Fritz et al. in DNA Cloning, ed by Glover (Oxford, UK: IRC Press, 1985)). M13 is a plasmid and transformation system in itself, and an ideal sequencing vector. M13 can be grown on Rec-strains of E. coli. The M13 genome is expandable (Messing et al. in The Single-Stranded DNA Phages, eds Denhardt et al. (NY: CSHL Press, 1978) pages 449453; and Fritz et al., supra) and M13 does not lyse cells. Extra genes can be inserted into M13 and will be maintained in the viral genome in a stable manner.

Many techniques for “displaying” biomolecules on the surface of phage are described in the art, and in general involves constructing a bacteriophage that expresses and displays at its surface the desired molecule (in this case an immunogenic enhancer) as a fusion product, while allowing the phage to remain intact and infectious.

Bacteriophage Lambda for Multicomponent Display and Vaccine Development

The present invention includes strategies to use phage for the display of multiple vaccine antigen epitopes on the phage head (capsid) surface. The DNA fragments coding for the vaccine antigen epitopes are fused in-frame to outer phage capsid proteins. The fusion proteins, containing both the amino acid sequences of the antigen epitopes and the normal phage capsid protein sequences, are assembled onto the phage capsids.

The phage vectors also contain a genomic construct coding for the vaccine antigen epitope(s), which is/are under the control of the ubiquitous cytomegalovirus promoter (CMV). This construct is cloned into a non-essential genome region of the phage vaccine vector. This recombinant phage vaccine offers a high-density foreign antigen epitope display on its surface, as well as carrying the gene(s) for the foreign antigen epitope(s) operably linked to a eukaryotic promoter, such that the epitope(s) will be expressed in the targeted professional immune cells. Moreover, this construct provides for any posttranslational modifications that may be of importance for a robust immune response in the vaccinated mammalian subject. An example of the construction of an efficacious multicomponent vaccine based on this system is provided in the Examples below.

As an alternative strategy, the present invention also contemplates the use of two separate populations of recombinant phage: one set of phage that are either native phage or are engineered to express one or more immune enhancers on its coat (or which will be expressed through the use of mammalian promoters in the host's immune cells), and the other set containing phage that express the pathogenic antigens on their coat, or that through the use of mammalian promoters are expressed in the host cells. This strategy would involve a vaccination protocol comprising a priming of the immune system with the first set of phage, which is subsequently followed by immunization with the second set of phage.

Vaccine Compositions

Compositions suitable for vaccination with the recombinant phage of the invention are prepared by admixing the recombinant phage with conventional excipients, i.e., pharmaceutically acceptable organic or inorganic carrier substances suitable for parenteral, enteral (e.g., oral) or topical application and that do not deleteriously react with the phage.

Suitable pharmaceutically acceptable carriers include but are not limited to water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the phage. They can also be combined where desired with other vaccines to make a polyvalent vaccine, but a polyvalent vaccine can also exist as a plurality of phage expressing different antigenic epitopes.

Vaccination

The vaccine compositions according to the present invention are administered in a conventional manner, for example, intramuscularly or subcutaneously, at a dose of approximately 10¹¹ pfu to 10⁸ pfu phage.

Enhancing the immune response through activation of immune regulator such as antigen presenting cells (e.g. dendritic cells, macrophages, B-lymphocytes) are known in the art. Thus, in another aspect, the present invention can harness the power of anamnestic response of the immune system of mammalians to prime any immune regulators, but particularly the antigen presenting cells, prior to active immunization against a specific antigen or antigens. The specific steps used for this aspect are: (1) native phage or genetically engineered ‘immune enhancer’ phage (about 10¹¹ pfu to 10⁸ pfu) are injected into the host either intramuscularly (I/M) or subcutaneously (S/C). This will activate all immunoregulators, mainly those of antigen presenting cells, against the phage; (2) after 14 days, the same host is injected with the same species of phage (about 10¹¹ pfu to 10⁸ pfu) but which is genetically engineered to display one or more specific antigens of interest on it's surface. Due to the prior immune priming with same virus, the uptake and processing of this recombinant phage will be enhanced several magnitudes by specialized antigen processing cells. During this process phage-display antigens will be co-processed with high efficiency along with phage proteins, thus activating immunity against such antigens with high efficacy. Subsequent booster vaccinations with the recombinant phage are optionally administered.

The invention is further described in the following non-limiting examples.

### EXAMPLES

### Example 1

Vaccine Platform Construction Outline

The basic platform vaccine construction outlined below can be used as the basis of an almost unlimited number of different vaccines. Briefly, to construct the phage vector vaccine of the present invention, one first selects an antigen believed to be useful for eliciting a certain protective immune response (for example, the rabies glycoprotein, which serves as a functional component of the rabies vaccine). The gene for this antigen is then cloned and placed as two copies in a bacteriophage: one of the two copies is constructed with a mammalian promoter (to be expressed as a vaccine gene in a professional immune cell in a mammalian host), while the other copy of the antigen gene is placed in the phage with a bacterial promoter as a fusion product with one of the major coat proteins of the phage (so that the fusion product is displayed on the recombinant phage coat).

More particularly, an antigen of a disease-causing microbe is selected which is believed to be capable of eliciting a protective immune response in a vaccinated subject. The gene for this antigen may be amplified (from the original source microbe) using, for instance, the polymerase chain reaction (PCR). Another option to obtain a sufficient quantity of the gene is to directly synthesize the gene (along with ensuring the presence of appropriate restriction sites for subsequent cloning into a vector).

The amplified gene is then cloned into a plasmid, for example a commercially available plasmid such as pCMV-Script® Vector (available from Stratagene, LaJolla, Calif.). The pCMV-Script® plasmid contains a strong mammalian promoter (CMV) derived from cytomegalovirus. The reason for this cloning step is to place the antigen gene in operable proximity to the CMV promoter such that the expression of the antigen gene will occur once transfected into a mammalian cell at vaccination.

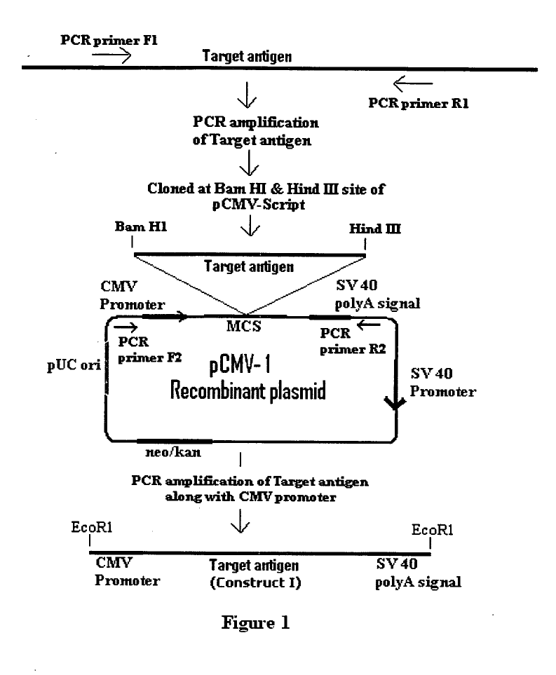

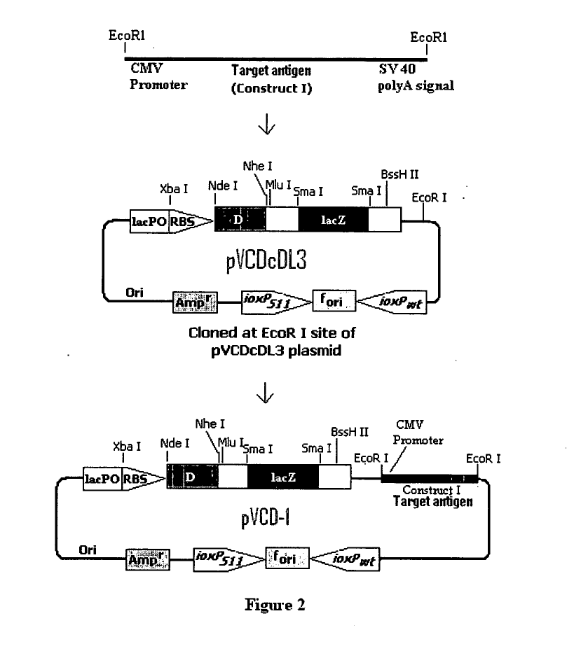

The region of the resulting plasmid (“pCMV-I”) containing the CMV promoter, the cloned antigen gene, and an SV40 polyA signal, is amplified by PCR using appropriate primers. This antigen gene/CMV promoter construct (designated as construct 1 in FIG. 1) is then cloned into the EcoR1 site of plasmid pVCDcDL3 (see FIG. 2) (GenBank accession no. AY190304). A schematic diagram of this recombinant plasmid is depicted in FIG. 2.

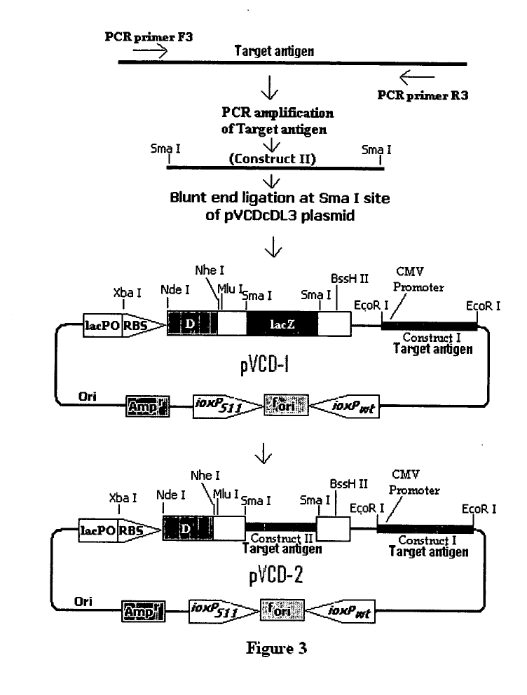

A second copy of the antigen gene obtained from the source microbe is amplified and cloned into the phage vector such that it will be translated and transcribed in a bacterial host as a fusion product with a major phage coat protein gene. The PCR primers used-for this second antigen gene amplification are choosen so that the amplification product will contain SmaI restriction sites (alternatively, the antigen sequence can be directly synthesized and include such restriction sites). This construct is depicted as construct II in FIG. 3. Following the amplification, the PCR product (or synthesized product) is digested with SmaI restriction enzymes. The resulting fragment is ligated into plasmid pVCD-I (previously digested with SmaI) to create a D protein fusion that will be displayed on the surface of the phage. The resulting plasmid will thus contain two copies of the antigen gene: one associated with a mammalian promoter and the other as a fusion gene with a gene for a major coat protein of the phage (the D capsid gene). This construct is depicted in FIG. 3.

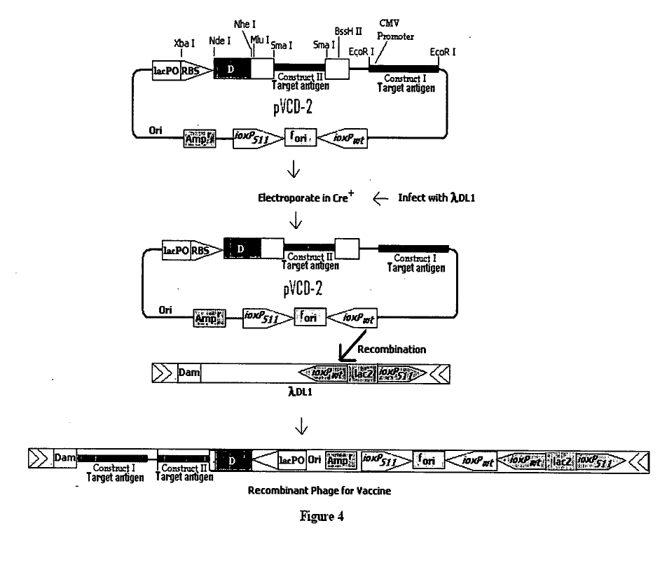

This plasmid is then electroporated into a Cre(+) strain of E. coli, grown in media containing ampicillin, and is infected with a compatible phage, such as lambda phage DL1. Lambda phage DL1 contains the construct IoxPwt-lacZalpha-loxP511 inserted into its genome between genes J and Int. Recombination will occur between the lox sites of the plasmid and lox sites of phage DL1, resulting in the introduction of plasmid DNA into the phage genome. See FIG. 4.

After sufficient time in culture to allow for recombination to occur, cell-free lysate is obtained and used to infect a culture of Cre(−) strain of E. coli, which is then plated on LB agar containing ampicillin. Colonies that grow on Amp agar are those in which the phage vector construct has integrated into the lambda DNA in the presence of Cre protein (supplied by Cre+ host strain), thereby conferring ampicillin resistance.

Amp resistant Cre(−) colonies containing the lambda integrate are grown at 37° C. until spontaneous lysis occurs. The cell free supernatant is used to infect Cre(−) E. coli cells to produce plaques. Single plaques are amplified by the liquid lysis method, and further purified by PEG-NaCl precipitation followed by CsCl density centrifugation.

### Example 2

The rabies glycoprotein gene (GenBank Accession No. X71879) is amplified by reverse transcriptase-PCR (RT-PCR) followed by a conventional PCR using forward primer F1 and reverse primer R1 from the original vaccine strain. Sequences of the primers:

F1: (^{5′} AGGATCCATGGTTCCTCA^{3′})
R1: (^{5′} GGGAAGCTTAATTCAGGA^{3′})

The synthesized glycoprotein gene is digested with BamH1 and HindIII in the appropriate buffers. pCMV-Script® is digested with BamH1 and HindIII in the appropriate buffers. Purified insert (MinElute® PCR Purification Kit, Qiagen) and vector (digested plasmid run on agarose gel, vector purified by MiniElute Gel Extraction Kit, Qiagen) are ligated together for 1 hour at room temperature using T4 DNA ligase, and the ligation mixture is used to transform E. coli strain XL1-Blue using conventional procedures. Transformed cells are incubated overnight at 37° C. on LB agar with kanamycin 40 ug/ml (LB Kan). Single colonies are picked and examined for rabies glycoprotein DNA insert by PCR (T3 and T7 primers are used—supplied as part of pCMV-Script® Vector cloning kit).

The resulting PCR fragments are run on an agarose gel to confirm correct fragment size (˜1.7 kb). This plasmid is designated pCMV-I (FIG. 1).

Plasmid pCMV-I is PCR amplified using the following primers (restriction sites underlined):

CMV For-ATGAATTCTGATTCTGTGGATAAC (F2 primer);
and
CMV Rev-TAGAATTCGATACATATTTGAATGTATT (R2 primer).

The resulting ˜3.3 kb fragment (containing CMV promoter, rabies glycoprotein gene, and polyA signal sequence) is purified (MinElute PCR Purification Kit, Qiagen), and digested with EcoR1 in appropriate buffer.

Plasmid pVCDcDL3 is digested with EcoR1 in appropriate buffer, and ligated with EcoR1 digested insert CMV-glycoprotein-polyA using T4 DNA ligase for 1 hour at room temperature. The ligation mixture is used to transform E. coli strain XL1-Blue by conventional methods. Transformed cells are plated on LB amp 100 ug/ml, and incubated overnight at 37° C. Single colonies are picked and inoculated into LB amp broth (100 ug/ml), and grown overnight at 37° C. Plasmid DNA is isolated (QIAprep Spin Mini Kit, Qiagen), and digested with EcoR1. Aliquots are examined by agarose gel electrophoresis. The presence of two bands (3.4 kb and 1.7 kb) indicates successful cloning. This plasmid is designated pVCD-I (FIG. 2).

Synthesized rabies glycoprotein gene containing SmaI site is digested with the same enzymes in appropriate buffers and purified as described above.

pVCD-1 is digested with SmaI in the appropriate buffers, and vector DNA is purified by agarose gel electrophoresis (MiniElute® Gel Extraction Kit, Qiagen). Digested pVCD-I is ligated to SmaI digested glycoprotein insert by T4 DNA ligase at room temperature for 8 hours. The ligation mixture is used to transform E. coli strain XL1-Blue by conventional methods. Transformed cells are plated on LB amp 100 ug/ml, and incubated overnight at 37° C. Single colonies are picked and inoculated into LB amp broth (100 ug/ml), and grown overnight at 37° C. Plasmid DNA is isolated (QIAprep® Spin Mini Kit, Qiagen) and restriction junctions of the insert are sequenced to confirm proper orientation of gene. This plasmid is designated pVCD-2 (FIG. 3).

Cre(+) E. coli strain BM 25.8 (Novagen, Madison, Wis.) is transformed by pVCD-2 and grown in LB amp broth (100 ug/ml) at 37° C. to OD₆₀₀ 0.3. About 1×10⁸ cells are harvested by centrifugation, and suspended in 100 μl of lambda phage DL1 lysate at an MOI of 1.0. After incubation at 37° C. for 10 minutes, the sample is diluted in 1 ml LB amp (100 ug/ml) +10 mM MgCl₂, and growth continues with shaking at 37° C. until lysis occurs.

A cell free lysate from the preceding step is prepared by filtration (0.22 um filter, Milipore), and 100 ul of the lysate is added to 200 ul of a log phase culture of Cre(−) E. coli strain TG1. This mixture is allowed to incubate for about 20 minutes at 37° C., and spread onto LB amp (100 ug/ml) plates. Plates are incubated overnight at 37° C.

Amp resistant Cre(−) colonies are inoculated into 5 ml LB amp broth (100 ug/ml), and incubated at 37° C. with shaking until lysis occurs (˜4 hours). The resulting lysates are filtered through 0.22 um filters (Milipore). 100 μl phage lysate is incubated with 200,p of a log phage culture of TG1 for 20 minutes at 37° C. The mixture is combined with 0.8% LB top agar, and poured onto LB plates. Plates are incubated overnight. Well isolated single plaques are picked and amplified by the liquid lysis method. (The liquid lysis method is a process where phage infection is propagated in liquid environment, and is often used for large scale phage production. Generally, host bacteria (here E. coli) is grown in suitable media (in this case, LB media) at 37° C. up to 0.2 OD prior to infection with phage. Three multiplicity of infection (moi) is used to assure the infection of each bacterium in the culture. Phage-bacterial infection is further propagated at 37° C. until visible lysis of bacterial debris is observed in the culture media. At this stage, OD generally drops below 0.02 and the culture is harvested for further purification through a cesium density gradient.)

### Example 3

Method for Cloning Dendritic Cell Targeting Peptide

To amplify a dendritic cell-targeting peptide (ATYSEFPGNLKP), two phosphorylated oligonucleotides are synthesized:

Den 1: 5′-GCGACCTATTCTGAATTTCCGGGCAACCTGAAACCG
Den 2: 5′-CGGTTTCAGGTTGCCCGGAAATTCAGAATAGGTCGC

100 μM oligo Den 1 is combined with 100 uM Den 2, T4 DNA ligase buffer (final concentration 1×), and water to a final volume of 10 ul. The mixture is heated to 95° C. for 5 minutes, the allowed to cool to room temperature.

Vector pVCDcDL3 is digested with SmaI in the appropriate buffer and purified by agarose gel electrophoresis (MiniElute Gel Extraction Kit, Qiagen). The purified vector, the annealed Den 1/Den 2 fragment, and water are combined to a total volume of 17 μl. 2 μl 5× T4 DNA ligase buffer, and 1 μl T4 DNA ligase are added for a final reaction volume of 20 μl. The ligation reaction is allowed to proceed overnight (about 16 hrs) at 4° C.

The ligation mixture from the preceeding step is used to transform E. coli strain XL1-Blue by conventional procedures, and then spread on LB amp (100 μg/ml) agar. Plated are allowed to incubate overnight at 37° C.

Single amp resistant colonies are picked and PCR amplified using the primers:

Den 3: 5′-tggcagcggagctagcaacg
Den 4: 5′-cattaaatgtgagcgagtaa

The resulting PCR fragment (˜675bp) is purified (MinElute® PCR Purification Kit, Qiagen) and sequenced to determine correct orientation of insert.

### Example 4

Construction of a Phage Vector Vaccine for IBDV

The VP2 protein of IBDV is selected as the immunogenic component for this vaccine.

Primers F1/R1 are employed for the amplification of the cDNA of the VP2 gene using the polymerase chain reaction (PCR) from the plasmid plBDVP2. The F1 primer (5′-TGMGGATCCTATGACGMCCTGCM-3′) is synthesized to correspond to nucleotides 131-145 of segment A of the IBDV genome and contain a BamHI restriction site (depicted in italics in the F1 primer sequence).

The R1 primer (5′ATTTAAGCTTCTATAGTGCCCGMTTATGTCCTT-3′) is synthesized to correspond to nucleotides 1463-1480 of segment A and it contains a HindIII restriction site (in italics) and a TAG termination codon (in bold). The length of the amplified VP2 cDNA is approximately 1350 bp.

The amplified gene is then cloned into a commercially available plasmid (pCMV-Script® Vector obtained from Stratagene). (This plasmid contains a strong ubiquitous cytomegalovirus promoter (CMV) derived from cytomegalovirus.) The synthesized VP2 gene is digested with BamH1 and HindIII in the appropriate buffers. pCMV-Script is also digested with BamH1 and HindIII in the appropriate buffers. Purified VP2 insert (MinElute® PCR Purification Kit, Qiagen) and vector (digested plasmid run on agarose gel, vector purified by MiniElute® Gel Extraction Kit, Qiagen) are ligated for 1 hour at room temperature using T4 DNA ligase, and the ligation mixture is then used to transform E. coli strain XL1-Blue using conventional procedures.

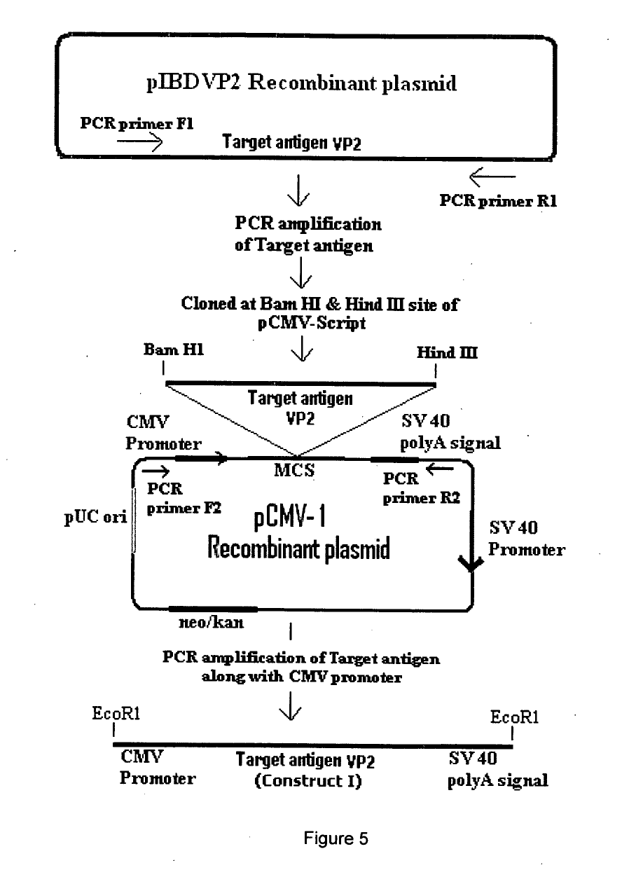

Transformed cells are incubated overnight at 37° C. on LB agar Kan (kanamycin 40 ug/ml). Single colonies are picked and examined for VP2 DNA insert by PCR (T3 and T7 primers are used as supplied as part of pCMV-Script® Vector cloning kit). The resulting PCR fragments are run on an agarose gel to confirm the correct fragment size (˜1.7 kb). This plasmid is designated pCMV-I (FIG. 5).

The region of plasmid pCMV-I containing the CMV promoter, the cloned VP2 gene, and an SV40 polyA signal are amplified by PCR using modified primers F2 and R2:

CMV For-ATGAATTCTGATTCTGTGGATAAC (F2 primer);
and
CMV Rev-TAGAATTCGATACATATTTGAATGTATT (R2 primer).

The resulting ˜3.3 kb fragment (containing CMV promoter, VP2 genes, and polyA signal sequence) is purified (MinElute® PCR Purification Kit, Qiagen), and digested with EcoR1 in the appropriate buffer.

Plasmid pVCDcDL3 (Gene bank accession no. AY190304) is digested with EcoR1 in the appropriate buffer, and ligated with the EcoR1-digested CMV-VP2 -polyA insert from above using T4 DNA ligase for 1 hour at room temperature. The ligation mixture is used to transform E. coli strain XL1-Blue by conventional methods.

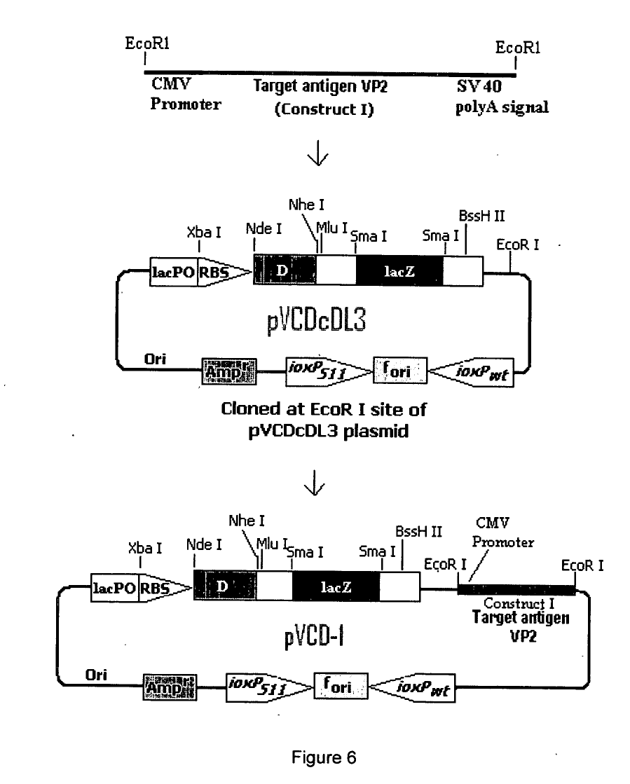

Transformed cells are plated on LB amp (ampicillin 100 μg/ml), and incubated overnight at 37° C. Single colonies are picked and inoculated into LB amp broth (100 ug/ml), and grown overnight at 37° C. Plasmid DNA is isolated (QIAprep Spin Mini Kit, Qiagen), and digested with EcoR1. Aliquots are examined by agarose gel electrophoresis. The presence of two bands (3.4 kb and 1.7 kb) indicates successful cloning. This plasmid is designated pVCD-I (FIG. 6).

A second copy of the VP2 gene is cloned such that it will be translated and transcribed in a bacterial host as a fusion product with a major phage coat protein gene. The PCR primers used for this second copy of antigen epitope coding gene amplification contain SmaI restriction sites and the resulting amplified product will thus include restriction sites for SmaI. Primers F3/R3 are employed to amplify the cDNA of the VP2 gene by polymerase chain reaction (PCR) from the source plasmid plBDVP2. The F3 primer:

(5′-TGAAGGGCCCTATGACGAAC CTGCAA-3′)

is synthesized according to nucleotides 131-145 of segment A and contains a SmaI restriction site (depicted in italics). The R3 primer:

(5′ATTTCCCGGGTATAGTGCCCGAATTATGTCCTT-3′)

is synthesized according to nucleotides 1463-1480 of segment A and contains a SmaI restriction site (depicted in italics).

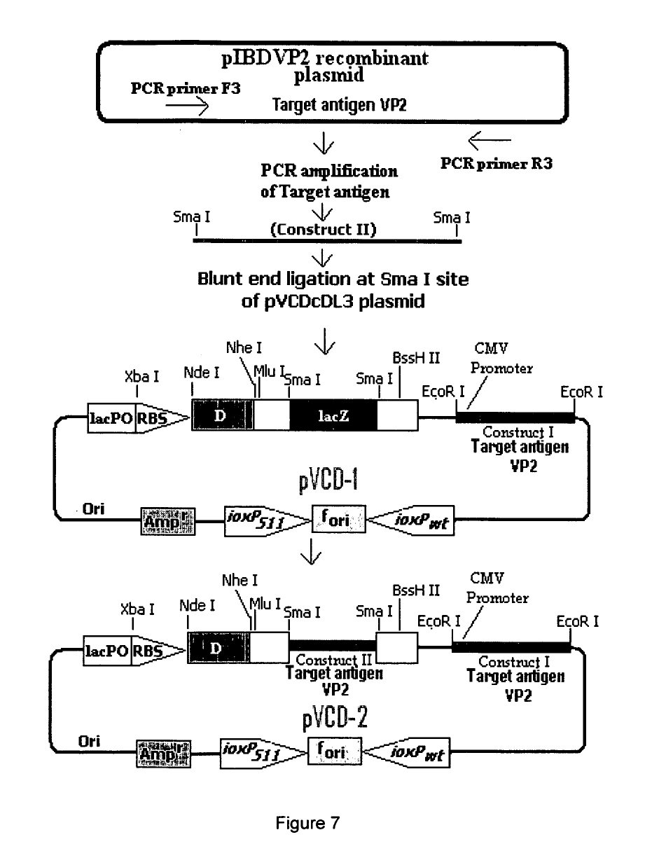

This construct is depicted as construct II in FIG. 7. Following the amplification, synthesized VP2 gene containing SmaI sites is digested with the same enzymes in the appropriate buffers and purified as described above. pVCD-1 is also digested with SmaI in the appropriate buffers, and plasmid vector DNA is purified by agarose gel electrophoresis (MiniElute® Gel Extraction Kit, Qiagen).

Digested pVCD-1 is ligated to SmaI digested the VP2 insert by T4 DNA ligase at room temperature for 8 hours. The ligation mixture is used to transform E. coli strain XL1-Blue by conventional methods. Transformed cells are plated on LB amp (ampicillin 100 μg/ml), and incubated overnight at 37° C. Single colonies are picked and inoculated into LB amp broth (100 μg/ml), and grown overnight at 37° C.

Plasmid DNA is isolated (QIAprep Spin Mini Kit, Qiagen) and restriction junctions of the insert are sequenced to confirm proper orientation of gene. This plasmid is designated pVCD-2 (FIG. 7). The recombinant plasmid thus contains two copies of the VP2 gene, one associated with a mammalian promoter and the other as a fusion with a gene for a major coat protein of the phage (the D capsid gene). This construct is depicted in FIG. 7.

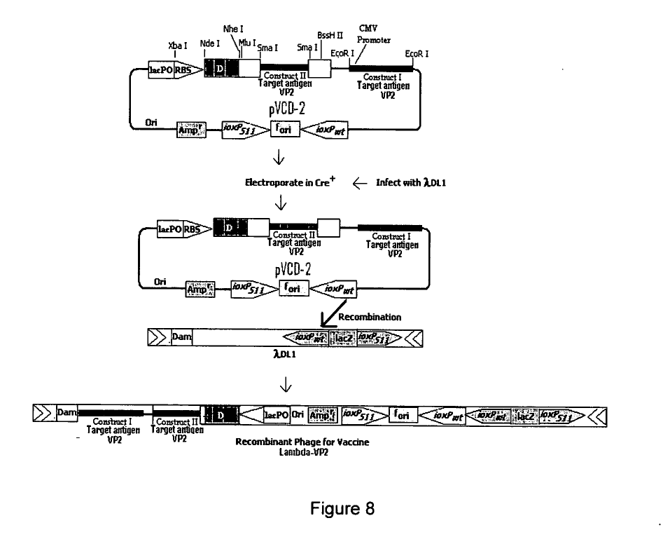

The recombinant pVCD-2 plasmid is then electroporated into a Cre(+) strain of E. coli, grown in media containing ampicillin, and is infected with lambda phage DL1. Lambda phage DL1 contains the construction loxPwt-lacZalpha-loxP511 inserted in its genome between genes J and Int. Recombination will occur between the lox sites of the plasmid and lox sites of phage DL1, resulting in the introduction of plasmid DNA into the phage genome. See FIG. 8. In particular, Cre(+) E. coli strain BM 25.8 (Novagen, Madison, Wis.) is transformed by pVCD-2 and grown in LB amp broth (100 ug/ml) at 37° C. to OD₆₀₀ 0.3. About 1×10⁸ cells are harvested by centrifugation, and suspended in 100 μl of lambda phage DL1 lysate at an MOI of 1.0. After incubation at 37° C. for 10 minutes, the sample is diluted in 1 ml LB amp (100 μg/ml) +10 mM MgCl₂, and growth continues with shaking at 37° C. until lysis occurs.

Cell free lysate obtained after the above recombination event is used to infect a Cre(−) strain of E. coli, and is plated on LB agar containing ampicillin. More particularly, a cell free lysate from the preceeding step is prepared by filtration (0.22 μm filter, Milipore), and 100 μl of the lysate is added to 200 μl of a log phase culture of Cre(−) E. coli strain TG1. This mixture is allowed to incubate for about 20 minutes at 37° C., and spread onto LB amp (100 μg/ml) plates. Plates are incubated overnight at 37° C. Colonies that grow on Amp agar are those in which the VP2 vector construct has integrated into the lambda DNA in the presence of Cre protein (supplied by Cre(+) host strain), thereby conferring ampicillin resistance.

Amp resistant Cre(−) colonies are inoculated into 5 ml LB amp broth (100 μg/ml ampicilin), and incubated at 37° C. with shaking until lysis occurred (about 4 hours). The resulting lysates are filtered through 0.22 μm filters (Milipore). 100 μl phage lysate is incubated with 200 μl of a log phage culture of TG1 for 20 minutes at 37° C. The mixture is combined with 0.8% LB top agar, and poured onto LB plates. Plates are incubated overnight.

Well isolated single plaques are picked and amplified by the liquid lysis method. Lysate is further purified by PEG-NaCl precipitation followed by cesium chloride (CsCl) density gradient centrifugation.

SDS-PAGE and Western blot analysis of recombinant Lambda-VP2 protein: Sodium dodecylsulfate polyacrylamide gel electrophoresis (SDS-PAGE) is carried out when the phage concentration has reached 10¹⁰ phages/ml. After SDS-PAGE analysis, the recombinant VP2 is verified by Western blot with the monoclonal antibody (mAb) R63 raised against the vaccine of the D78 strain (ATCC VR-2047).

Antigen-capture enzyme-linked immunosorbent assay (AC-ELISA) detection of VP2 expression on Lambda-VP2: mAbs raised against the vaccine of the D78 strain (ATCC VR-2047) and chicken sera against the virulent strain of IBDV are used in AC-ELISA to examine their immunoreactivy to the recombinant lambda-VP2, the wild-type lambda, and a virulent IBDV strain. The AC-ELISA procedure is performed as reported previously in Lim B L, Cao Y C, Yu T, Mo C W., J. Virol. 1999, 73: 2854-2862.

### Example 5

Mouse Inoculation Study

Mice are inoculated with the phage vaccine vector described in Example 4, lambda-VP2, to confirm the specific immune response against lambda-VP2 recombinant.

Fifteen 30 day-old Balb/C female mice are randomly divided into three groups, each of 5 mice. The mice are raised in isolators with sterilized water and feed. Group 1 is inoculated subcutaneously with lambda-VP2 phage in an oil emulsion vaccine at day 0, and given a booster with the same vaccine intramuscularly at day 7. Group 2 is immunized with wild-type lambda phage in an oil emulsion, while Group 3 is administered only a saline oil emulsion. Each immunizing dose of phage for one mouse contains about 2×10⁹ phage.

Blood is collected from the inner canthus of eye socket and allowed to coagulate naturally. Prior to inoculation all mice in the study are tested for lambda antibody (previous experiments with mice have demonstrated no detectable native antibody to lambda phage). The serum is separated by the conventional methodology and stored at −20° C. A commercial IBD ELISA kit (IDEXX, Westbrook, USA) is used to assess the IBD antibody in the sera. Specific mouse conjugate is used instead of the kit conjugate to perform the ELISA. A positive result with the lambda-VP2 phage vaccine is indicative of successful protection against IBDV.

### Example 6

Vaccination of Chickens with Lambda-VP2

Thirty-day old white leghorn chickens are randomly divided into three groups, each with 10 chickens. The chickens are raised in isolators with sterilized water and feed.

Group 1 is inoculated subcutaneously with the lambda-VP2 phage in an oil emulsion vaccine at day 14 postnatal, and given a booster with the same vaccine intramuscularly at day 28 postnatal.

Group 2 is immunized with wild-type lambda phage in an oil emulsion, while Group 3 is administered a saline oil emulsion. Each immunizing dose of phage for one chicken contains about 2×10⁹ phages.

Blood is collected and allowed to coagulate naturally. The serum is separated by the conventional method and stored at −20° C. A commercial IBD ELISA kit (IDEXX, Westbrook, USA) is used to assess the IBD antibody in the sera.

At day 52 postnatal, each group are infected by the virulent IBDV at a LD₅₀. The numbers of sick and dead birds are recorded 7 days post-infection. At the end of the experiment, all surviving birds are weighed and euthanized. The bursa of each chicken is then weighed. The body weight/bursa weight is used to calculate the B/B value. A positive result is evaluated on the basis of complete protection (survival rate after challenge) and B/B index. The bursa index will be accounted for the ratio of the B/B value of the testing group and that of the control group. A high bursa index for chickens immunized with T4-VP2 will signify a positive result.

### Example 7

Vaccination through Ova Administration and Challenge Infection

Thirty 18-day-old fertilized hen's eggs are randomly divided into three groups, each with 10 eggs. Group 1 is inoculated with lambda-VP2 phage vaccine suspended in phosphate buffer saline (PBS). Group 2 is administered wild-type lambda phage suspended in PBS, and Group 3 is only administered PBS.

Each immunizing dose with phage for one egg will contain about 2×10⁸ phages. Eggs are injected with 0.1 ml of lambda-VP2 vaccine/PBS into the large end of the egg, which contains the air cell, with a fine needle. The 18-day-old chicken embryo's immune system has been shown to be mature enough to respond efficiently to vaccination. The eggs are then transferred into the incubator hatchery where they remain until they hatch at about 21 days of age.

After hatching, blood is collected periodically from week-old chicks and allowed to coagulate naturally. The serum is separated by the conventional methodology and stored at −20° C. A commercial IBD ELISA kit (IDEXX, Westbrook, USA) is used to assess the IBD antibody in the sera.

At day 21 postnatal, each group is infected by the virulent IBDV at an LD₅₀ dose per chicken. The numbers of sick and dead birds are recorded 7-days post infection. All living birds at the end of the experiment will be weighed and then euthanized. The bursa of each chicken is will also be weighed. The body weight/bursa weight will be used to calculate the B/B value. A positive result will be evaluated on the basis of complete protection (survival rate after challenge) and B/B index. The bursa index will be accounted for by the ratio of the B/B value of the testing group and that of the control group. A high bursa index for chicken (hatched out from immunized eggs with T4-VP2) will signify positive result.

### Example 8

Construction of a Phase Vector Vaccine for Rapid Development and Application ('Shotgun Approach’):

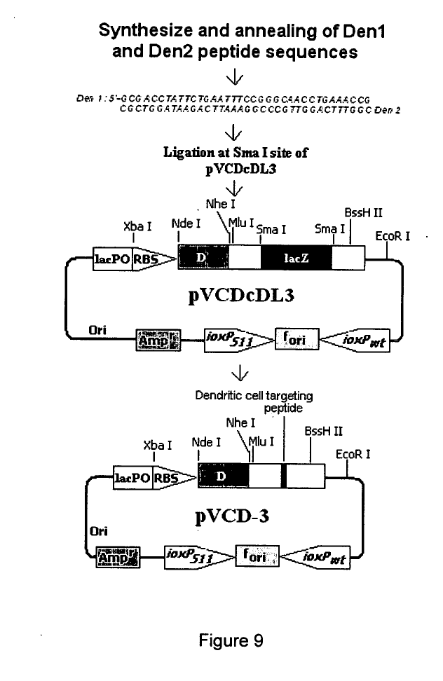

The construction of a lambda phage containing a dendritic cell-targeting peptide is performed using the methods described in Example 3 (see also FIG. 9).

Construction of λ Phage Expression Vector Containing CMV and T7 Dual Promoters.

The pDUAL GC Mammalian Expression Vector (pDUAL GC 6.6 kD, Stratagene, Calif.) expresses proteins containing a C-terminal c-myc epitope tag, which is derived from the human c-myc gene and contains 10 amino acid residues (EQKLISEEDL). The c-myc epitope tag is well-characterized and is highly immunoreactive (although any selectable tag may be used). High level gene expression in mammalian cells is achieved using the human cytomegalovirus immediate early promoter/enhancer (CMV IE). Inducible gene expression in prokaryotes is obtained using the hybrid T7/lacO promoter, whereby expression is regulated using isopropyl-β-D-thio-galactopyranoside (IPTG) in bacteria that contain T7 RNA polymerase. Two modified primers, each containing a Mfe1 restriction site (underlined) at it's 5′ end sequence designated as “CMV forward”

“CMV forward”
5′ ATACCGCAATGAAAGGTTTTGCGCCATTC3′
(F2.1 primer)
and
“CMV reverse”
5′ AACGCCAATTGTAACAAAATATTAACGCTTAC 3′
(R2.1 primer)

primers are used to amplify a DNA segment of ≈2.3 kb that contains a cytomegalovirus promoter, a T7 promoter, two unique Eam11041 restriction sites, an SV40 polyadenylation signal, a T7 terminator and a c-myc epitope tag. Amplified product is purified (MinElute PCR Purification Kit, Qiagen), and then digested with Mfe1.

Plasmid pVCDcDL3 (Genbank Accession No. AY1 90304) is digested with EcoR1 and ligated with the Mfe1—digested PCR amplified product of the plasmid's CMV polyA insert (Mfe1 and EcoR1 produce compatible 5′ overhangs). T4 DNA ligase is used for ligation and the reaction is continued for 1 hour at room temperature.

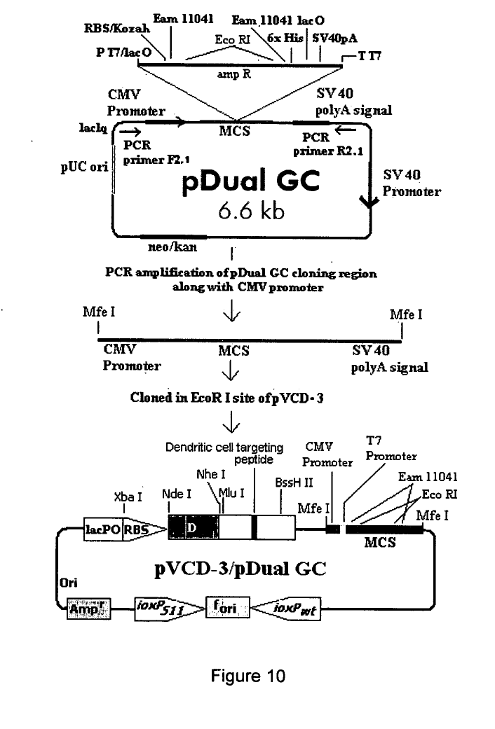

The ligation mixture is used to transform E. coli strain TG1, Cre(−) (supEΔhsdM-mcrB)5(r-K mk-McrB-)thiΔ(lac-proAB) [F/traD36, LaclqΔ(lacZ)M15]) by conventional methods. Transformed cells are plated on LB broth with 100 μg/ml ampicillin (“LB amp”), and incubated overnight at 37° C. Single colonies are selected and inoculated into LB amp broth, and grown overnight at 37° C. Plasmid DNA is isolated (QIAprep Spin Mini Kit, Qiagen), and digested with Mfe1. Aliquots are examined by agarose gel electrophoresis. The presence of two bands (4.0 kb and 2.3 kb) indicates successful cloning. This plasmid is designated pVCD-3/pDual GC (FIG. 10).

Cloning of Organism DNA in pVCD-3/pDual GC Recombinant Plasmid.

The infectious agent of interest is grown in suitable media or cell culture and harvested in a conventional manner, such as sucrose or cesium gradient, etc. Genomic DNA (which, in the case of an infectious agent having an RNA genome, cDNAs are prior synthesized by reverse transcriptase PCR) is collected with by the phenol-chloroform extraction method (Maniatis T., Fritsch E. F., Sambrook J. 1992. Molecular. Cloning: A Laboratory Manual. Cold Spring Harbor, N.Y.: Cold Spring Harbor Lab. Press) and restriction digested by using Sau3A1 site-specific endonuclease. Six μl of the DNA sample containing about 1.0 μg/μl of DNA are mixed with 36 μl of distilled water and 5.0 μl of 1× Sau3A1 digestion buffer [100 mM NaCl, 10 mM Tris-HCL, 10 mM MgCl2, (pH 7.3)], supplemented with 0.5 μl (100 μg/ml) bovine serum albumin. The content of the tube is gently mixed in an Eppendorf centrifuge at 10,000 rpm for five seconds. Finally, 2.5 μl of enzyme (10 units/μl) is added and the mixture is centrifuged at 10,000 rpm for five seconds in an Eppendorf centrifuge, and is kept at 37° C. in a water bath for one hour.

The reaction is stopped by the addition of EDTA to a final concentration of 25 mM. A small aliquot is electrophoresed over 1% agarose gel to monitor the digestion. One hundred μl of TE buffer is added to the mixture and the DNA is extracted once with phenol and subsequently washed three times with chloroform:isoamyl alcohol at the ratio of 24:1. The restriction digested DNA is precipitated in presence of ethanol.

Synthesis and Ligation of Adapters to Organism DNA Fragments.

Three different types, (1, 2, and 3) of Eam11041-BamH1 conversion adapters are prepared by the annealing of six different kinds of synthetic oligonucleotides, and each of these adapters is ligated separately to the Sau3A1 cohesive ends of the genomic DNA fragments of the organism.

Synthesis of Duplex Oligonucleotide Conversion Adapters.

Each oligonucleotide used to form the duplex conversion adapters is synthesized by and obtained from Oligos ET Inc. (Wilsonville, Oreg.). One strand (A strand) of each duplex conversion adapter contains the cohesive end (ATG) at the 5′ terminus to the 10 mer core annealing sequence (see FIG. 1). Three lengths of the “A strand” (A1, A2, and A3) are synthesized by the addition of single cytosine residues between the 5′ end of the core sequence and 3′ end of the ATG cohesive end. Oligonucleotides complimentary to each length of the “A strand” core annealing sequences (14 mer=B1, 15 mer=B2, 16 mer =B3) are synthesized with Sau3A1, Mbo1 or BamH1 cohesive termini (GATC) added to the 5′ end of the “B strand”.

The duplex conversion adapters are formed by separately annealing “A strands” and “B strands” with matching lengths of complimentary core sequences. To do this, a 0.5 A260 unit of each of the lyophilized oligonucleotides is dissolved in 120 μl of distilled water to obtain a 50 μM solution. Forty μl of each of these complimentary oligonucleotides (A1+B1, A2+B2, A3+B3) are mixed with 10 μl of 10× buffer (250 mM Tris, pH 8.0, 100 mM MgCl2) and 10 μl of distilled water. These mixtures are heated separately to 95° C. and slowly cooled (approximately one hour) to room temperature. This yields 20 μM solutions of 1, 2 and 3 types of adapters. At this point the three lengths of each of the duplex conversion adapters with identical cohesive ends may be stored separately at −80° C. for future use.

Ligation of Adapters to the DNA or cDNA Fragments of the Infectious Agent of Interest.

The Sau3A1 restriction fragments (6 μg) are dry ethanol precipitated and then re-suspended in 45 μl of distilled water and aliquoted in three equal parts (parts 1, 2 and 3) in Eppendorf tubes. Next, 15 μl of pre-annealed adapters type 1, 2 and 3 are added to parts 1, 2 and 3, respectively, to yield approximately a 10:1 molar ratio of adapter to the insert fragments. To each of these mixtures, 5.0 μl of 10× ligase buffer (500 mM Tris, pH 7.5, 70 mM MgCl2, 10 mM DTT), 0.5 μl of 10 mM ATP, 13 μl of distilled water, and 1.5 μl (6 Weiss units) of T4 DNA ligase (Stratagene, La Jolla, Calif.) are added, mixed well and incubated at 15° C. for six hours. After completion of this ligation reaction the contents of the three Eppendorf tubes are mixed together in one tube and are placed in a 70° C. water bath for 10 minutes to heat inactivate the ligase enzyme. Subsequently, the tubes are cooled on ice.

Phosphorylation of Adapter Modified Insert DNA and Removal of Excess Adapters

Adapter-modified insert DNA is prepared for ligation into pVCD-3/pDual GC by phosphorylation of adapter 5′ ends with T4 polynucleotide kinase (Promega Corporation, Madison, Wis.) and spin column chromatography is used to remove excess adapters. Following heat inactivation and cooling, 150 μl of the reaction mixture are added to 20 μl of 10× T4 polynucleotide kinase buffer (500 mM Tris-HCL, pH 7.5, 100 mM MgCl2, 50 mM DTT, 1.0 mM spermidine), 10 μl of 0.1 mM ATP, 1.0 μl of T4 polynucleotide kinase (10 units), and 19 μl of distilled water. The reaction mixture is incubated at 37° C. for 30 minutes and the reaction is terminated by single extraction with 1 volume of TE-saturated phenol, followed by three extractions of equal volume of chloroform:isomyl alcohol (24:1). The upper aqueous phase is transferred to a fresh tube and unligated adapters are efficiently removed with spin column chromatography.

The Sephacryl S400 matrix, spin columns, wash tubes and collection tubes for column chromatography are obtained from Promega Corporation (Madison, Wis.). The chromatography columns are prepared according to the instructions of the Promega technical bulletin (# 067). Briefly, Sephacryl S-400 slurry is thoroughly mixed and 1.0 ml slurry is transferred to a spin column. The column tip is placed in the wash tubes and then the whole assembly is placed inside a large centrifuge tube (Falcon #25319) and centrifuged in a swing bucket rotor at 800×g for five minutes. The wash tube with fluid in it is discarded, and a second centrifugation is performed in the same manner to discard any remaining fluid in the column. The phosphorylated reaction mixture with excess adapters is applied to the top of the gel bed of the prepared column and the column is placed into the collection tube. This whole assembly is then centrifuged in the same manner as described above in the column preparation step. The phosphorylated adapter-modified insert DNA present in the eluant of the collection tube is then ethanol precipitated at −20° C. overnight by adding 0.5 volume of 7.5 M ammonium acetate and 2.0 volumes of ethanol. The precipitated DNA is pelleted by centrifugation at 4° C. for 15 minutes and the invisible pellet is washed once with 70% alcohol prior to vacuum drying.

Ligation of Insert DNA to PVCD-3/pDual GC Plasmid.

The adapter-modified phosphorylated vacuum dried insert DNA pellet is suspended in 6.0 μl of TE (10 mM Tris, pH 8.0, 0.1 mM EDTA). The optimal vector:insert ratio for efficient ligation is obtained by aliquoting 2.5, 0.5 and 0.1 μl of the infectious agent insert DNA into three separate tubes. One μg of Eam11041-digested and dephosphorylated pVCD-3/pDual GC plasmid DNA is added to each of the tubes, followed by 1.0 μl of 10× ligase buffer, 0.1 μl of 10 mM ATP, and distilled water to 9.0 μl. Then 1.0 μl of T4 DNA ligase (4 Weiss units, Stratagene) is added and the solution incubated at 15° C. for six hours. The ligation mixture is used to transform E. coli strain TG1, Cre(−) (supEΔhsdM-mcrB)5(r⁻ₖmₖ⁻McrB⁻)thiΔ(lac-proAB) [F′traD36, Lacl^{q}Δ(lacZ)M15]) by conventional methods. Transformed cells are plated on LB broth containing 100 μg/ml kanamycin, and incubated overnight at 37° C. Single colonies are selected and inoculated into LB kanamycin (100 ug/ml) broth, and grown overnight at 37° C. Plasmid DNA is isolated (QIAprep Spin Mini Kit, Qiagen), and digested with Mfe1.

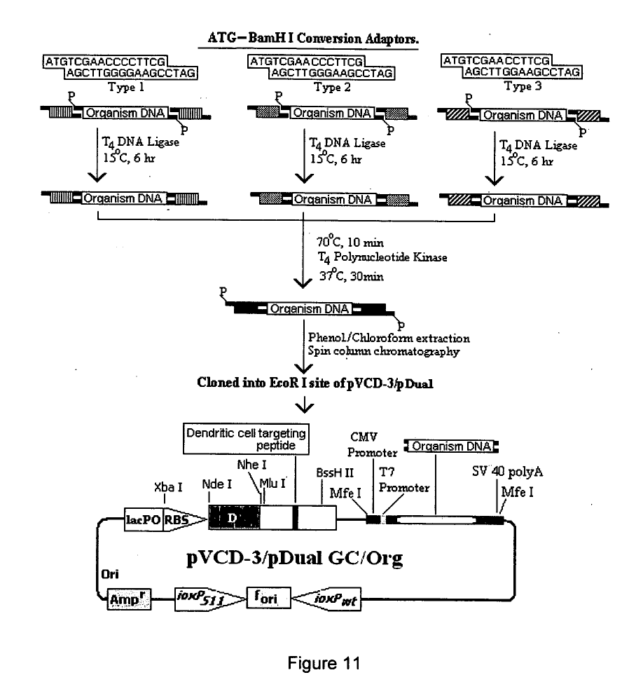

Aliquots are examined by agarose gel electrophoresis to confirm the ligation of insert DNA fragments. The new recombinant plasmid, designated as pVCD-3/pDual GC/Org plasmid (FIG. 11) is then electroporated into a Cre(+) strain of E. coli, grown in media containing ampicillin, which is infected with lambda phage DL1. Lambda phage DL1 contains the construct loxPwt-lacZalpha-loxP511 inserted in its genome between genes J and Int. Recombination will occur between the lox sites of the plasmid and lox sites of phage DL1, resulting in the introduction of plasmid DNA into the phage genome. In particular, Cre(+) E. coli strain BM 25.8 (Novagen, Madison, Wis.) is transformed by pVCD-2 and grown in LB amp broth (100 μg/ml) at 37° C. to OD₆₀₀ 0.3. About 1×10⁸ cells are harvested by centrifugation, and suspended in 100 μl of lambda phage DL1 lysate at an MOI of 1.0. After incubation at 37° C. for 10 minutes, the sample is diluted in 1 ml LB amp (100 μg/ml) +1 mM MgCl₂, and allowed to grow with shaking at 37° C. until lysis occurs.

Cell free lysate obtained after the above recombination event is used to infect a Cre(−) strain of E. coli, and then plated on LB agar containing ampicillin. More particularly, a cell free lysate from the preceeding step is prepared by filtration (0.22 μm filter, Milipore), and 1 00 μl of the lysate is added to 200 μof a log phase culture of Cre(−) E. coli strain TG1. This mixture is allowed to incubate for about 20 minutes at 37° C., and spread onto LB amp (100 μg/ml) plates. Plates are incubated overnight at 37° C. Colonies that grow on Amp agar are those in which the DNA fragments of the infectious agent have integrated into the lambda DNA in the presence of Cre protein (supplied by Cre(+) host strain), thereby conferring ampicillin resistance.

Amp resistant Cre(−) colonies are inoculated into 5 ml LB amp broth (100 μg/ml), and incubated at 37° C. with shaking until lysis occurs (about 4 hours). The resulting lysates are filtered through 0.22 μm filters (Milipore). 100 μl phage lysate is incubated with 200 μl of a log phage culture of TG1 for 20 minutes at 37° C. The mixture is combined with 0.8% LB top agar, and poured onto LB plates. Plates are incubated overnight. To obtain a high titer phage for storage, packaged recombinants are amplified by plating approximately 50,000 μlate forming units (pfu) and incubating at 37° C. for about 6 hours. When the plaques attain the size of about 0.5 mm, 10 ml of phosphate buffer is added to the plate and incubated overnight while shaking at 4° C. The suspension containing phage was extracted once with chloroform and stored in the presence of 0.3% chloroform.

Immunoscreening of Recombinant Phase for Expression of Antigens

The phage recombinant clones are screened for expression of antigens using rabbit anti-c-myc antibody available from Sigma-Aldrich (Cat# M4439). Screening the phage recombinants for expression of antigens is done according to the following procedures.

An E. coli strain expressing T7 polymerase is used as a host cell for recombinant phage screening. A liquid culture is started from a single colony and grown overnight with vigorous shaking at 30° C. in LB media supplemented with 0.2% maltose and 10 mM MgSO4. The cells are centrifuged at 1000×g for 10 minutes then gently resuspended in 0.5 volumes of 10 mM MgSO₄. About 700 to 1000 pfu of the phage recombinants are mixed with 1.2 ml of above prepared E. coli cells and incubated at 37° C. for 18 minutes. Twenty one ml of molten LB top agar (0.8%), prewarmed to 42° C. are then added, mixed, and poured onto a 150 mm plate containing 1.5% LB bottom agar and the agar is allowed to solidify at room temperature for 15 minutes. The plates are incubated at 37° C. for four hours, until the plaques are about one mm in size. Next, a 137 mm colony/plaque screen membrane (NEN® Research products, Boston, Mass.) is saturated with IPTG solution (10 mg/ml) and blotted dry on a filter paper. This membrane is carefully placed on the top agar and incubation is continued at 37° C. for another three hours. The membrane is pierced asymmetrically at three places with an 18 gauze needle, peeled from the agar, and washed three times with Tris saline to remove the debris and bacteria. The plates are then stored at 4° C. and the washed NEN membranes are blocked with casein solution at 4° C. overnight.

The next day, membranes are incubated in a 1:100 dilution of the anti-c-myc antibody for two hours at room temperature and washed twice in Tris saline with 0.05% Triton X-100, and once in Tris saline for 15 minutes each. The antibody treated membranes are incubated either with 2.0 μg/ml of alkaline phosphatase labeled goat anti-rabbit IgG or mouse anti-rabbit IgG (Kirkegaard and Perry) for one hour at room temperature. The membranes are consecutively washed three times in the same way described earlier in this procedure, followed by a final wash with 0.9% NaCl. Finally the membranes are treated with Fast Red and naphthol substrate solution for about 10 minutes and the reaction is stopped by washing the membrane in distilled water.

The pink immunoreactive spots corresponding to the recombinants expressing pathogen antigens are aligned with the help of the needle marks and those positive plaques were picked up from the plates with the aid of a Pasteur pipette. The agar plugs containing the recombinant plaques are dispensed separately into 500 μl of SM buffer and the phages are allowed to diffuse out by vortexing and incubating vials at 4° C. for two hours. Twenty μl of chloroform are also added separately in each vial for long term storage. Plaque purification of the recombinants is accomplished by two additional rounds of immunoscreening as above.

Well isolated single plaques are selected and amplified separately by the liquid lysis method. Lysates are mixed together to purify through PEG-NaCl precipitation and cesium chloride density gradient. Finally, purified recombinant phages are dialyzed extensively in PBS (pH4.0) to remove cesium chloride. At this stage, the phages expressing the multitude of proteins of the infectious agent of interest are ready to be used as a vaccine.

Optionally, these recombinant phages can be screened again in a eukaryotic system for protein expression (described more fully below). Human dendrite cells collected from peripheral blood monocytes are used for this purpose. This additional step allows further enrichment of the recombinant phage vaccine components by selecting those phages that express recombinant protein(s) of the infectious agent used in the method in antigen presenting cells.

Protein Expression Determination in Human Dendritic Cells.

Peripheral blood monocytes (PBMC) are isolated from peripheral blood of healthy donors by Ficoll-hypaque gradiant centrifugation. Monocytes are purified by using the MACS CD14 isolation kit (Miltenyi Biotec, Bergisch Gladbach, Germany). Subsequently, monocytes are cultured in six-well plates (0.5 to 1.5×10⁶ cells/ml) in fresh complete medium supplemented with 1000 U/ml GM-CSF and 500 U/ml IL4 and cells are harvested after a total culture period of 48 hours. Cells harvested from 48-hour cultures are distributed in 96 well plates (10⁶ cells/well) and infected with 107 phage particles (sterilized by filtration through 0.25 micrometer filter). After an additional 48 hours of incubation, cells are harvested and lysed by a freeze-thaw technique and then analyzed by Western blotting to confirm the specificity of the expressed proteins.

SDS-PAGE and Western Blot Analysis of Recombinant Lambda-Recombinant Proteins.

Sodium dodecylsulfate polyacrylamide gel electrophoresis (SDS-PAGE) is carried out in the conventional manner, and the recombinant proteins (expressed by separate clones) are verified by Western blot with the anti-c-myc antibody. A plurality of the recombinant lambda phages, determined by the above method as carrying the genes for the various antigens of the pathogen of interest that are capable of being expressed in the dendritic cells, will form the active components of the vaccine.

What is claimed is:

1. An infectious, recombinant phage that expresses (a) one or more immunogenic enhancer molecules, and (b) one or more peptides derived from a pathogen of interest which have an epitope that will induce immunological response in a mammalian host cell.

2. The phage of claim 1, wherein the epitope is contained within a protein of a pathogenic microbe.

3. The composition of claim 1, wherein the epitope of interest is contained within a VP2 protein of IBDV or the rabies glycoprotein.

4. The phage of claim 1, wherein the epitope is contained within a cancer-specific protein.

5. The phage of claim 4, wherein the cancer-specific protein is human aspartyl-asparaginyl hydroxylase (HAAH).

6. The phage of claim 1, wherein the one or more immunogenic enhancer molecules are expressed on the coat of the phage, and serve to target the delivery of the phage to a mammalian host's immune cells.

7. The composition of claim 1, wherein the immunogenic enhancer molecule is one that targets dendritic cells in the host.

8. The phage of claim 1, wherein the one or more peptides derived from a pathogen of interest are operably linked to a mammalian promoter, whereby expression will occur in a mammalian host cell.

9. A composition comprising a plurality of the infectious, recombinant phage of claim 1 in a pharamaceutically acceptable carrier.

10. A composition according to claim 9, wherein the plurality of phage comprise separate phages that express a multitude of different proteins of a single pathogen.

11. A method for preparing an infectious, recombinant phage, comprising:
inserting one or more genes encoding for immunogenic enhancer(s) into the phage genome, such that the phage will express the immunogenic enhancer(s) on its coat; and
inserting one or more genes coding for pathogenic immunogen(s) of interest under the control of a mammalian promoter, such that expression of said genes occurs in a mammalian host cell.

12. A method for inducing an immunogenic response to a pathogen of interest in a mammalian host, comprising administering a composition according to claim 9 to said mammalian host, whereby the mammalian host will mount an immunological response to the pathogen of interest.
